(19) United States
(12) Patent Application Publication
(10) Pub. No.: US 2009/0093735 A1
(43) Pub. Date: Apr. 9, 2009

(54) Test unit and test system for analyzing body fluids

(76) Inventors: Stephan Korner, Cham (CH); Brigitte Niederberger, Thalwil (CH); Denise Schnarrwyler, Luzern (CH)

Correspondence Address: ROCHE DIAGNOSTICS OPERATIONS INC., 9115 Hague Road, Indianapolis, IN, 46250-0457, US

(21) Appl. No.: 12/240,341

(22) Filed: Sep. 29, 2008

Related U.S. Application Data
(63) Continuation of application No. PCT/EP2007/022755, filed on Mar. 28, 2007.

(30) Foreign Application Priority Data
Mar. 29, 2006 (EP) 06006523.2

(51) Int. Cl. A61B 5/157; A61B 5/151; G01N 33/50

(52) U.S. Cl. 600/583; 600/573; 422/68.1; 436/180

(57) ABSTRACT

The test unit for analyzing body fluids such as blood comprises a collecting element which has a microfluidic collecting area for the body fluid, and a test element which can be brought into fluidic connection with the collecting area by deflecting a transfer part of the collecting element in order to transfer body fluid. The transfer part is pre-tensioned against a holding-down device and the deflection of the transfer part towards the test element can be triggered by releasing the holding-down device.

This application is a continuation of PCT/EP 2007/002755 filed Mar. 28, 2007 that claims priority to European Patent Application No. EP 06006523.2 filed Mar. 29, 2006, which are hereby incorporated by reference.

### FIELD

The disclosure concerns a test unit and a test system for analyzing body fluids such as blood.

### BACKGROUND

A collecting device for body fluids is known from WO 2005/084530 in which a movable part of a flow path for body fluid is actively brought into contact with a receiving, means for the fluid by means of a contacting means in that for example the movement is driven by a mechanical or magnetic actuator. This allows a small amount of fluid to be subjected to a processing in a test process at a defined time. With regard to the relevant prerequisites and advantages reference is explicitly made to the said document and the contents of which are herewith incorporated. However, the active contacting requires a drive unit in the device which increases the manufacturing costs and makes the process sequence more complicated. In particular care must also be taken that the interaction between the device drive and the moved element which is part of a disposable is sufficiently accurate.

### SUMMARY

On this basis the object of the disclosure is to further develop the units and systems known in the prior art such that a highly reliable controlled measurement process is made possible using simpler means.

Invention embodiments are based on the idea of enabling an external drive to be, omitted by using an inherent transfer movement. Accordingly it is proposed according to invention embodiments that the transfer part is pre-tensioned against a holding-down device (down-holder) and the deflection of the transfer part towards the test element can be triggered by releasing the holding-down device. The pre-tensioning allows an automatic movement between an initial state and a contact state where the initial state is maintained by the holding-down device as a retaining means until the triggering takes place. Only low precision requirements are needed for this because the movement of the transfer part as such requires no external control or guidance. Otherwise the already mentioned advantages of a triggering of the test process at a defined time are fully attained. This has a positive effect especially in the case of disposable articles in which very small amounts of sample (for example micro liters and less) are transferred from the collecting site to the site at which the test is carried out by micro fluidic processes and preferably by capillary action.

The collecting area extends at least partially over the transfer part, wherein the collecting area and the test element are physically separated at a distance from one another before the deflection and are in contact with one another after the deflection. In this manner the uptake of fluid can also take place under difficult conditions whereas a directed transfer of sample does not take place until the collecting process has been successfully completed.

A structurally embodiment provides that the transfer part is attached to the collecting element with a pre-bend, such as a bending arm.

For an inherent transport movement by deformation, the transfer, part can be deflected with a bending deformation by the action of inner stresses and/or surface tensions. This can be achieved by means of the fact that the transfer part has a bending stress which causes the deflection due to a different surface treatment on different sides. Another possibility is that the transfer part can be deflected due to different layers of material like a bimetallic strip. The manufacturing process is made particularly simple when the transfer part is formed from a flat substrate by an etching process where different etching depths on different sides of the transfer part result in inner bending stresses in the transfer part. In this manner no additional bending process is required because the bending pre-stress is already obtained by a process step of a profiling process that is suitable for mass production.

It is also possible that the transfer part can be deflected by means of plastic deformation. Such a deformation can be carried out in the production process as a pre-bending or also not until it is used in the device.

Another embodiment provides that the collecting element has a lancing member for insertion into a body part for example into the finger and the collecting area can be supplied with body fluid via the puncture.

In order to fix the initial state of the transfer part in a simple manner, the holding-down device can be formed by a separable bridge of material between the collecting element and the transfer part in which case the deflection of the transfer part can be triggered by cutting through the bridge of material. This can be accomplished particularly simply by forming the collecting element and the holding-down device in one piece from a flat material in particular a sheet of high-grade steel where the holding-down device is formed by at least one severable cross-piece.

Alternatively it is also possible without any particular problems in the manufacture for the holding-down device to be formed by an adhesive strip or a laminate mounted on the collecting element and stretching over a section of the transfer part.

In order to prevent a premature transfer of liquid when the test element in the initial state, a spacer can be included on the collecting element at a preset distance from the transfer part. The collecting element and the transfer part advantageously form a constructional unit that is intended to be a disposable single-use product.

In order to enable a directed transfer even of the smallest amounts of sample, the transfer part in the contact state can engage with the test element while maintaining a preset contact angle.

The test element can have a reagent field to which body fluid can be applied through, an opening of the collecting area and in particular an opening on the longitudinal side of a capillary channel.

Embodiments of the invention also encompasses a test system for examining body fluids such as blood in which the transfer part is pre-tensioned against a holding-down device on the collecting element where the deflection can be triggered by releasing the holding-down device by means of a trigger in the device.

In this connection, it is possible that the trigger is formed by a severing unit that operates without contact and in particular a laser. Another embodiment provides that the trigger has a mechanically active cutting means. It is also conceivable, that the trigger is formed by a thermal actuator for triggering a bending deformation under the action of heat.

The test element can be handled or positioned in the system independently of the collecting element especially in order to transfers a quantity of liquid onto a separate measuring position.

### BRIEF DESCRIPTION OF THE DRAWINGS

The invention is elucidated in more detail in the following on the basis of the embodiment examples shown schematically in the drawings.

FIG. 1 shows a block diagram of a test system with a collecting element and test element for taking up and examining blood embodiment.

FIG. 2 shows a perspective view of the collecting element with a transfer part for the blood sample in a pre-bent state embodiment.

FIG. 3 shows a simplified side-view and top-view of the collecting element in FIG. 2 embodiment.

FIG. 4 shows a diagram of the collecting element corresponding to FIG. 3 and additionally with the holding-down device and test element in the initial state embodiment.

FIG. 5 shows the collecting element of FIG. 4 in the triggered state embodiment.

FIGS. 6 and 7 show diagrams of a collecting element with a separating cross-piece as a holding-down device embodiments corresponding to FIGS. 4 and 5.

### DETAILED DESCRIPTION

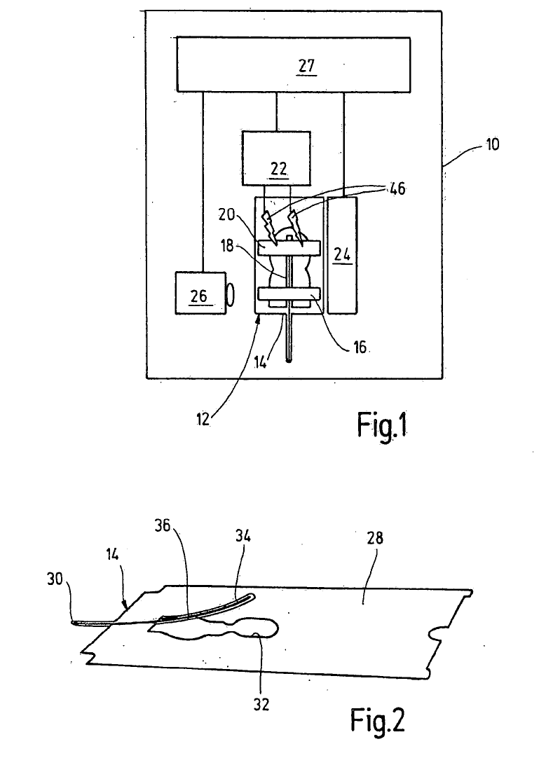

The test system 10 embodiment shown in FIG. 1 is designed as a portable blood glucose measuring device and enables the use of disposable test units 12 for the self-determination of blood glucose by patients. For this purpose each test unit 12 comprises a collecting element 14 for taking up a microscopic amount of blood and a test element 16 which can be loaded with blood by deflecting a transfer part 18 of the collecting element 14 where in its initial state the transfer part 18 is kept at a distance from the test element 16 by a holding-down device 20.

Further system elements can include a trigger 22 for the holding-down device 20, a lancing drive 24 for the collecting element 14 and a measuring unit 26 for a preferably optical scanning of the test element 16. A processor unit 27 provides for instrument process control and evaluation of the measuring results.

As shown in FIG. 2, the collecting element 14 is formed in one piece by etching from a flat substrate 28 and in particular a sheet of high-grade steel. In this process a distally projecting, needle-shaped lancing shaft 30 is formed on the substrate part 28 whereas a pre-bent transfer part 34 is cut free in the area of a central substrate opening 32. A groove-shaped semi-open capillary channel 36 runs continuously between the lancing shaft 30 and the transfer part 34 in order to transport the blood sample obtained by a puncture in a body part.

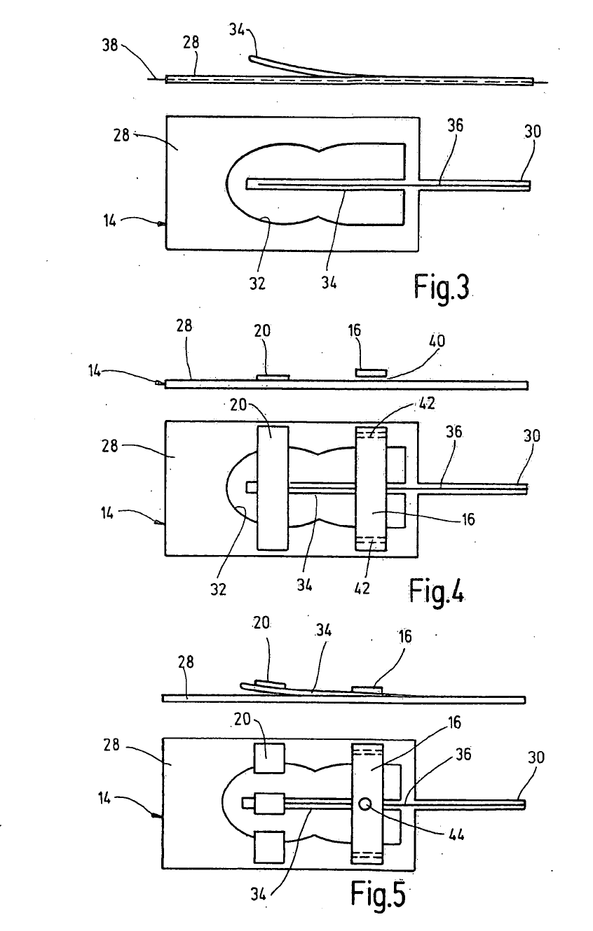

As shown in FIG. 3, the transfer part 34 is bent out of the plane 38 of the substrate 28 as a curved bending arm, and namely towards the side on which the side of the channel 36 is open. The pre-bending can for example be due to the fact that the bending arm 34 undergoes a bending deformation under the action, of internal stresses and/or surface tensions. Particularly in the etching process different etching depths or asymmetric etching on the opposing substrate sides of the transfer part 34 can result in permanent internal bending stresses. Another method is a one-sided physical or chemical surface modification or treatment. It, is, also conceivable that a two-layer strip is formed by laminating an additional foil layer onto the transfer part 34 which due to different linear coefficients of expansion can be deflected under the action of heat like a bimetallic strip.

According to FIG. 4, the transfer part 34 is kept in the substrate plane 38 by a detachable holding-down device 20. The holding-down device 20 can for example be an adhesive strip which is glued onto the substrate 28 and spans the opening 32 and the proximal section of the transfer part 34 in such a manner that the bend in the transfer part 34 is held down. In this manner a clearance 40 to the test element 16 is kept clear in the initial state as a result of which there is initially no fluidic connection between the collecting channel 36 and the facing side of the test element 16. Hence, the air gap 40 prevents the direct transfer of blood fluid collected in the collecting channel 36 onto the test element 16.

The test element 16 can be formed by a reagent strip which is coated with dry chemicals which react to an analyte (glucose) in a body fluid (blood, optionally also tissue fluid) for example by a colour change thus enabling an optical detection. The reagent strip 16 is carried by spacers 42 in the area, of the substrate 28 in order to create a clearance 40 to the held-down transfer part 34.

As shown in FIG. 5, the holding-down device 20 can be released or severed by means of the trigger 22 such that due to the impressed pre-stressing or pre-bending, the transfer part 34 is laterally deflected at a defined trigger time and the channel 36 is brought into fluidic contact with the test element 16. This can be brought about by means of the fact that the trigger 22 controls a laser 46 (FIG. 1) as the severing unit in order to cut through the holding-down device 20 on both sides of the transfer part 34. As a result the transfer part curves automatically upwards such that a contact state with the test element 16 is reached and blood fluid is transferred as a sample spot 44 via the channel opening on the longitudinal side. Due to the bend in the transfer part 34, the engagement is reached while maintaining a preset contact angle which enables a local targeted transfer of blood with a high positioning accuracy.

Instead of a non-contact severing unit 46, a mechanical cutting means can also be provided to sever the holding-down device 20. It is also possible that a holding-down device laminated over the transfer part 34 has two functions by, on the one hand, acting as a retainer and, on the other hand, by causing a deflection due to thermal activation or the action of heat like a bimetal.

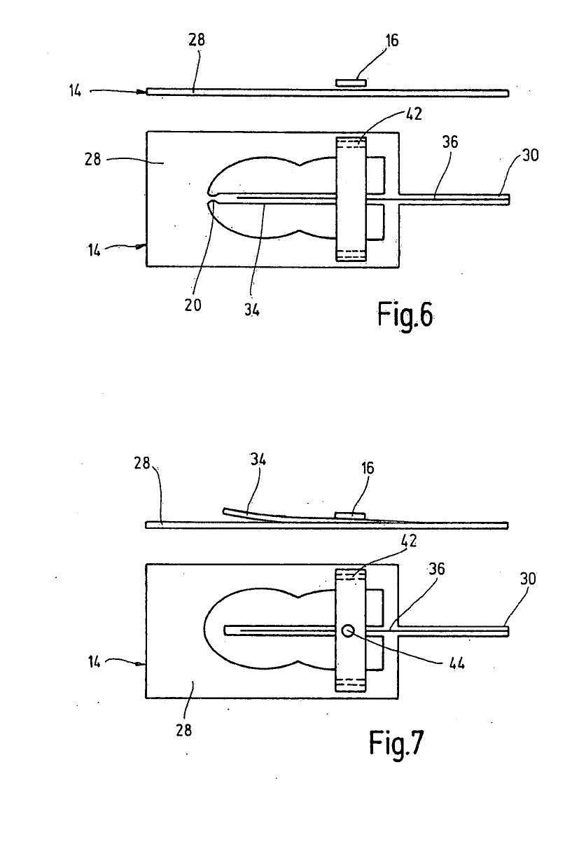

In the embodiment shown in FIGS. 6 and 7, the same or similar parts are labelled with the same reference numerals as described above. The holding-down device is in this case formed by a severable bridge of material in the form of a cross-piece 20 between the substrate 28 and the proximal end of the transfer part 34. This cross-piece 20 can be obtained during the manufacturing process by not completely etching through the substrate 28 so that no additional components or production steps are necessary. The triggering then takes place by severing this remaining connection 20 while the transfer part 34 curves into the impressed bent shape (FIG. 7).

When a test is carried out, a test unit 12 is activated from a device magazine in order to collect body fluid by a skin puncture by means of the lancing drive 24 in a reciprocating lancing movement. Fluid is then transported in the channel 36 by capillary action until the collecting area in the transfer part 34 is also sufficiently filled. From there the sample 44 can be transferred onto the test element 16 by the device triggering whereby constant measuring conditions for each test are created by the defined triggering time. After evaluation by means of the processor 27, the result of the measurement is displayed to the user and the used test unit 12 is disposed of as a disposable product.

Thus, embodiments of the test unit and test system for analyzing body fluids are disclosed. One skilled in the art will appreciate that the teachings can be practiced with embodiments other than those disclosed. The disclosed embodiments are presented for purposes of illustration and not limitation, and the invention is only limited by the claims that follow.

What is claimed is:

1. A test unit for analyzing body fluids such as blood, comprising:
a collecting element which has a microfluidic collecting area for a body fluid in the form of a semi-open channel; and,
a test element configured to be brought into fluidic connection, with the collecting, area by deflection of a transfer part of the collecting element in order to transfer the body fluid,
wherein the transfer part is pre-tensioned against a holding-down device and the deflection of the transfer part towards the test element is triggered by releasing the holding-down device.

2. The test unit in claim 1 wherein the collecting area extends at least partially over the transfer part, and the collecting area and the test element are physically separated at a distance from one another before the deflection and the collecting area and the test element are in contact with one another after deflection.

3. The test unit in claim 1 wherein the transfer part is attached to the collecting element with a pre-bend.

4. The test unit in claim 3 wherein the pre-bend is a bending arm.

5. The test unit in claim 1 wherein the transfer part can be deflected with, a bending deformation by the action of internal stresses and/or surface tensions.

6. The test unit in claim 5 wherein the transfer part has a bending stress which causes the deflection due to a different surface treatment on different sides.

7. The test unit in claim 5 wherein the transfer part can be deflected due to different material layers like a bimetallic strip.

8. The test unit in claim 1 wherein the transfer part is formed from a flat substrate by an etching process where different etching depths on different sides of the transfer part result in inner bending stresses in the transfer part.

9. The test unit in claim 1 wherein the transfer part can be deflected by means of plastic deformation.

10. The test unit in claim 1 wherein the collecting element has a lancing member for insertion into a body part and the collecting area can be supplied with body fluid via the puncture.

11. The test unit in claim 1 wherein the holding-down device can be formed by a severable bridge of material between the collecting element and the transfer part, and that the deflection of the transfer part can be triggered by cutting through the bridge of material.

12. The test unit in claim 1 wherein the collecting element and the holding-down device are formed in one piece from a flat material in particular a sheet of high-grade steel where the holding-down device is formed by at least one severable cross-piece.

13. The test unit in claim 1 wherein the holding-down device is formed by an adhesive strip or a laminate mounted on the collecting element which stretches over a section of the transfer part.

14. The test unit in claim 1 wherein the test element in the initial state is preferably arranged on the collecting element at a preset distance from the transfer part by means of a spacer.

15. The test unit in claim 1 wherein the collecting element and the transfer part form a constructional unit that is intended to be a disposable single-use product.

16. The test unit in claim 1 wherein the transfer part in the contact state engages with the test element while maintaining a preset contact angle.

17. The test unit in claim 1 wherein the test element has a reagent field, to which body fluid can be applied through an opening of the collecting area.

18. A test system for analyzing body fluids such as blood, comprising:
a device in which at least one collecting element can be used in order to detect an analyte in a body fluid;
a collecting element which has a microfluidic collecting area for the body fluid in the form of a semi-open channel; and,
a test element configured to be brought into fluidic connection with the collecting area by deflecting a transfer part of the collecting element in order to transfer body fluid;
wherein the transfer part is pretensioned against a holding-down device on the collecting element where the deflection can be triggered by releasing the holding-down device by means of a trigger in the device.

19. The test system in claim 18, wherein the trigger is formed by a serving unit that operates without contact and in particular a laser.

20. The test system in claim 18, wherein the trigger has a mechanically active cutting means.

21. The test system in claim 18, wherein the trigger is formed by a thermal actuator.

22. The test system in claim 18, wherein the test element is handled independently of the collecting element.

23. A test unit for analyzing body fluids such as blood, comprising:
a collecting element that has a microfluidic collecting area configured as a semi-opened channel for collecting the body fluid;
a test element carried on the collecting element for analyzing the body fluid;
means for transfer pretensioned against a holding-down device to deflect the collecting element to transfer body fluid to the test element; and,
means for triggering to release the holding-down device upon being triggered to allow deflection of the collecting element in order to transfer body fluid free from an external drive to deflect the collecting element.

24. A method for analyzing blood to determine blood glucose level, comprising:
pretensioning a transfer part of a collecting element against a holding-down device;
lancing a body part to provide access to blood;
collecting the blood in a semi-open channel microfluidic collection area;
triggering the holding-down device to release the holding-down device;
deflecting the collecting element free from use of an external drive to deflect the collecting element;
transferring the blood from the collection area to a test element;
analyzing the blood by the test element to determine blood glucose level.
